(19) United States
(12) Patent Application Publication
(10) Pub. No.: US 2006/0194200 A1
(43) Pub. Date: Aug. 31, 2006

(54) High throughput detection of glutathione s-transferase polymorphic alleles

(76) Inventors: Charles Keller, SALT LAKE CITY, UT (US); Linda Ballard, Salt Lake City, UT (US); Richard Lemmons, Salt Lake City, UT (US); Francis Ali-Osman, Houston, TX (US)

Correspondence Address: MADSON & AUSTIN;GATEWAY TOWER WEST, SUITE 900, 15 WEST SOUTH TEMPLE, SALT LAKE CITY, UT, 84101, US

(21) Appl. No.: 10/530,512

(22) PCT Filed: Oct. 15, 2003
(86) PCT No.: PCT/US03/32765

§ 371 Date: Apr. 6, 2005

Related U.S. Application Data
(60) Provisional application No. 60/418,876, filed on Oct. 15, 2002.

(51) Int. Cl. C12Q 1/68; C12P 19/34

(52) U.S. Cl. 435/6; 435/91.2

(57) ABSTRACT

A high-throughput assay for characterizing a subject's genetic makeup is disclosed. Specifically, a high-throughput assay utilizing PCR is disclosed that permits the rapid and accurate characterization of a subject's inherited alleles of the polymorphic glutathione S-transferase (GST) genes GSTM₁, GSTM₃, GSTP₁, and GSTT₁. This method allows detection of the specific alleles inherited, including the gene dosage of GSTM₁ and GSTT₁ while not requiring restriction endonuclease digestion of the PCR products in order to detect length differences. Further, the method allows all analyses to be performed simultaneously in the same gel lane, thus further adding efficiency and cost-effectiveness.

### RELATED APPLICATIONS

This application is related to and claims the benefit of U.S. Provisional Patent Application No. 60/418,876 filed Oct. 15, 2002, and entitled High Throughput Detection of Glutathione S-Transferase Polymorphic Alleles, which is incorporated herein by reference.

### STATEMENT REGARDING FEDERALLY SPONSORED RESEARCH

This invention was made with Government support under GCRC Grant Number M01-RR00064 from the National Center for Research Resources of the National Institutes of Health. The Government has certain rights to this application.

### BACKGROUND OF THE INVENTION

The present invention relates to a high throughput assay for genotyping glutathione S-transferase (GST). More specifically, the present invention relates to a high throughput assay for detecting the presence of clinically-significant GST polymorphic alleles in a patient. Such information may be subsequently used to predict the response of a cancer to a specified chemotherapy treatment regime.

Glutathione S-transferases (“GSTs”) are a family of enzymes which detoxify a wide range of molecules. More specifically, GSTs add sulfur to suitable acceptor molecules in the form of glutathione. It has been discovered that many environmental and non-environmental carcinogens, including halogenated and nitro compounds, organophosphates, alkylating agents, epoxides, and polycyclic aromatic hydrocarbons (such as those from cigarette smoke) are generally suitable substrates for this reaction. Davies et al., J. Clin. One., 19:5, 1279-1287 (2001). Completion of the reaction often detoxifies the substrate compound, and routes it through the gamma-glutamyl cycle for degradation. Id.

In addition to the above, polymorphisms of GST proteins have been correlated with altered risk of many cancers. The actions of the GSTs have also been observed to affect the response of a patient to common chemotherapeutic agents. Further, GSTs have been shown to affect the toxicity of many such chemotherapeutic compounds in cancer patients. Specific affects have been observed with alkylating agents such as chlorambucil, cyclophosphamide, melphalan, nitrogen mustard, and thiotepa; intercalating agents such as doxorubicin; and mitomycin C and carmustine. Id.

The GST family has been categorized into 4 main subclasses: GSTM1, GSTM3, GSTP1, and GSTT1. Each of these exhibits marked hereditary differences in substrate specificity and enzyme activity. Polymorphic alleles for each of these genes are summarized in Table 1, which is discussed in greater detail below. These GST polymorphic alleles occur at frequencies that range from at least 2.4-20%, to as much as 40-84% inheritance.

Chemotherapy is a primary treatment for many types of cancer. As chemotherapy has expanded in use, it has been observed that specific regimens and individual chemotherapeutic agents are more effective in some individuals than others. Similarly, some chemotherapeutic agents exhibit greater toxicity in individual patients than they do in most of the population being treated. As noted above, it has been demonstrated that some of these effects may be due at least in part to the presence or absence of particular polymorphisms of the GST alleles in the patient. In one specific example of this, it has been demonstrated that the overexpression of GSTP1 is associated with increased tumor grade, increased tumor stage, reduced patient survival and chemotherapy resistance. Current testing processes are slow, expensive, and fail to differentiate GSTM1 polymorphisms. Similarly, known methods fail to discover the gene dosage of GSTT1. Known methods are labor- and time-intensive, and thus are not useful in settings such as high-throughput screenings.

It would thus be an improvement in the art to provide high throughput methods for detecting GST polymorphisms. It would be a further improvement to provide such methods suitable for use with large patient groups, such as clinical pharmacogenetic trials. Such methods are provided herein.

### BRIEF SUMMARY OF THE INVENTION

The method of the present invention has been developed in response to the present state of the art, and in particular, in response to the problems and needs in the art that have not yet been fully solved by currently available GST assays. Thus, the present invention provides high throughput assays for the detection of GST polymorphisms and use of such methods in evaluating the potential toxicity of chemotherapy in a patient.

The invention first provides a high throughput assay for detecting GST alleles present in a patient. Such an assay may be useful for projecting the success of a specific chemotherapeutic therapy in a patient. Further, such an assay may be useful for evaluating the toxicity of a potential chemotherapeutic therapy in a patient. In some embodiments, the assays of the invention comprise the steps of obtaining a biological sample from the patient, isolating genomic DNA from the sample, performing PCR amplification of a portion of the DNA to detect GSTM1 alleles, performing PCR amplification of a portion of the DNA to detect GSTM3 and GSTT1 alleles, performing PCR amplification of a portion of the DNA to detect GSTP1 polymorphisms, and detecting GSTM1, GSTM3, GSTT1, and GSTP1 polymorphic alleles in the DNA obtained from the PCR amplification steps.

In the assays of the invention, the biological sample may be obtained from a wide variety of source materials. In some specific examples, the biological sample may be obtained from a peripheral blood sample. In others, the biological sample may be obtained from a Guthrie card. Further, the biological sample may alternately be obtained from buccal epithelial cells. It is known in the art that buccal epithelial cells may be obtained in a variety of ways, including from mouthwash used and expectorated by a patient, and from buccal swabs properly used in the mouth of a patient. Other sample sources known to one of skill in the art may also be used within the scope of the instant invention.

In the methods of the invention, the step of performing PCR amplification of a portion of the DNA to detect GSTM1 alleles may include performing fluorescent, allele-specific PCR on the portion of the DNA using GSTM1-specific primer sequences. In some methods of the invention, individual GSTM1-specific primer sequences separately include the sequences of SEQ ID NO: 2, SEQ ID NO: 3 and SEQ ID NO: 5. Some specific methods may include the GSTM1-specific primer sequences of SEQ ID NO: 1, SEQ ID NO: 3 and SEQ ID NO: 4. In this step, β-actin may be co-amplified along with the GSTM1 alleles to serve as a reaction control using B3-actin-specific PCR primer sequences. In these methods, β-actin-specific primer sequences may be used. Such primer sequences may separately include SEQ ID NO: 6 and SEQ ID NO: 8. In specific methods, these β-actin primer sequences may be SEQ ID NO: 6 and SEQ ID NO: 7.

According to the invention, the step of performing PCR amplification of a portion of the DNA to detect GSTM3 and GSTT1 alleles may include performing PCR amplification of a portion of the DNA using GSTM3- and GSTT1-specific primer sequences. In some methods of the invention, individual GSTM3- and GSTT1-specific primer sequences may separately include SEQ ID NO: 10, SEQ ID NO: 12, SEQ ID NO: 24 and SEQ ID NO: 26. Some specific suitable GSTM3- and GSTT1-specific primer sequences may be SEQ ID NO: 9, SEQ ID NO: 11, SEQ ID NO: 24 and SEQ ID NO: 25.

The step of performing PCR amplification of a portion of the DNA to detect GSTP1 polymorphisms in the methods of the invention may include performing fluorescent, allele-specific PCR amplification of a portion of the DNA using GSTP1-specific primer sequences. In some methods of the invention, suitable individual GSTP1-specific primer sequences may separately include SEQ ID NO: 14, SEQ ID NO: 16, SEQ ID NO: 18, SEQ ID NO: 20, SEQ ID NO: 22 and SEQ ID NO: 23. Some specific suitable GSTP1-specific primer sequences may be SEQ ID NO: 13, SEQ ID NO: 15, SEQ ID NO: 17, SEQ ID NO: 19, SEQ ID NO: 21 and SEQ ID NO: 23.

The methods of the invention next include a step of detecting GSTM1, GSTM3, GSTT1, and GSTP1 polymorphic alleles in the DNA obtained from the above-described PCR amplification steps. According to the invention, this detection step may first include combining the DNA obtained from the PCR amplification steps to detect GSTM1, GSTM3, GSTT1, and GSTP1 alleles. This saves time and cost by allowing detection methods to be performed on a single sample instead of on multiple samples. Multiple-sample detection may be performed within the scope of the invention if beneficial, however. The detection step may include electrophoresis of the combined DNA to allow detection of the individual alleles present using PCR product size differences and fluorescent tag differences. Gel and capillary electrophoresis are examples of possible electrophoresis techniques that may be used in the detection step of the methods of the invention.

The methods of the invention described above may be extended to allow gene dosage of GSTM1 alleles to be determined. More specifically, in the methods of the invention, a long range PCR assay of a portion of the DNA may be conducted to distinguish GSTM1*A/A or GSTM1*B/1 homozygotes from GSTM1*A/null and GSTM1*B/null heterozygotes. The long range PCR assay of a portion of the DNA may be conducted using GSTM1*0-specific primer sequences. In some specific examples, the GSTM1*0-specific primer sequences may be SEQ ID NO: 27 and SEQ ID NO: 28.

Similarly, the methods of the invention may be extended to allow determination of GSTT1 gene dosage. In such extended methods, a long range PCR assay of a portion of the DNA may be conducted to determine the gene dosage of GSTT1. The long range PCR assay of a portion of the DNA to determine the gene dosage of GSTT1 may be conducted using GSTT1*0-specific primer sequences. In some specific examples, the GSTT1*0-specific primer sequences may be SEQ ID NO: 33 and SEQ ID NO: 34. This step may alternatively be conducted using GSTT1*0-specific primer sequences and GSTT1/GSTT2-non-specific primer sequences. In some specific examples, the GSTT*0-specific primer sequences may be SEQ ID NO: 33 and SEQ ID NO: 34 and the GSTT1/GSTT2-non-specific primer sequences may be SEQ ID NO: 31 and SEQ ID NO: 32.

In a specific high throughput assay of the invention, the step of performing PCR amplification of a portion of the DNA to detect GSTM1 alleles includes using primers having the sequences of SEQ ID NO: 1, SEQ ID NO: 3 and SEQ ID NO: 4; the step of performing PCR amplification of a portion of the DNA to detect GSTM3 and GSTT1 alleles includes using primers having the sequences of SEQ ID NO: 9, SEQ ID NO: 11, SEQ ID NO: 24 and SEQ ID NO: 25; and the step of performing PCR amplification of a portion of the DNA to detect GSTP1 polymorphisms includes using primers having the sequences of SEQ ID NO: 13, SEQ ID NO: 15, SEQ ID NO: 17, SEQ ID NO: 19, SEQ ID NO: 21 and SEQ ID NO: 23.

In alternate embodiments of the method of the invention, the steps of performing PCR amplification of a portion of the DNA are followed by the steps of identifying portions of the DNA which failed PCR amplification and performing single nucleotide extension verification assays to obtain the information originally sought by the PCR amplification. In this manner, a sample may be completely processed despite failure in the initial steps of the high throughput methods.

As briefly noted above, the methods of the invention may include methods of assessing the potential toxicity of chemotherapy in a patient. Such assay methods generally comprise the steps of obtaining a biological sample from the patient, isolating genomic DNA from the sample, performing PCR amplification of a portion of the DNA to detect GSTM1 alleles, performing PCR amplification of a portion of the DNA to detect GSTM3 and GSTT1 alleles, performing PCR amplification of a portion of the DNA to detect GSTP1 polymorphisms, detecting GSTM1, GSTM3, GSTT1, and GSTP1 polymorphic alleles in the DNA obtained from the PCR amplification steps, and comparing the GSTM1, GSTM3, GSTT1 and GSTP1 polymorphic alleles present to predetermined standards to evaluate the potential toxicity of chemotherapy to the patient.

As in the general allele detections methods discussed above, the toxicity assessment methods exhibit sample versatility. More specifically, the biological sample may be obtained from peripheral blood; blood spotted onto filter paper such as Guthrie cards or other suitable products (Isocode®, Schleicher & Schuell, Keene, N.H.); and buccal epithelial cells obtained by methods such as expectorated mouthwash or buccal swabs.

Similarly, as in the general methods above, in the toxicity assessment methods of the invention, the step of performing PCR amplification of a portion of the DNA to detect GSTM1 alleles may include performing fluorescent, allele-specific PCR on the portion of the DNA using GSTM1-specific primer sequences. In some methods of the invention, individual GSTM1-specific primer sequences separately include the sequences of SEQ ID NO: 2, SEQ ID NO: 3 and SEQ ID NO: 5. Specific methods may include the GSTM1-specific primer sequences of SEQ ID NO: 1, SEQ ID NO: 3 and SEQ ID NO: 4. β-actin may be amplified with the GSTM1 alleles to serve as a reaction control. In these methods, β-actin-specific primer sequences may be used. Such primer sequences may separately include SEQ ID NO: 6 and SEQ ID NO: 8. In specific methods, these β-actin primer sequences may be SEQ ID NO: 6 and SEQ ID NO: 7.

In the toxicity assessment methods, the step of performing PCR amplification of a portion of the DNA to detect GSTM3 and GSTT1 alleles may include performing PCR amplification of a portion of the DNA using GSTM3- and GSTT1-specific primer sequences. In some methods of the invention, individual GSTM3- and GSTT1-specific primer sequences may separately include SEQ ID NO: 10, SEQ ID NO: 12, SEQ ID NO: 24 and SEQ ID NO: 26. Some specific suitable GSTM3- and GSTT1-specific primer sequences may be SEQ ID NO: 9, SEQ ID NO: 11, SEQ ID NO: 24 and SEQ ID NO: 25.

The step of performing PCR amplification of a portion of the DNA to detect GSTP1 polymorphisms in the toxicity assessment methods of the invention may include performing fluorescent, allele-specific PCR amplification of a portion of the DNA using GSTP1-specific primer sequences. In some such methods, suitable individual GSTP1-specific primer sequences may separately include SEQ ID NO: 14, SEQ ID NO: 16, SEQ ID NO: 18, SEQ ID NO: 20, SEQ ID NO: 22 and SEQ ID NO: 23. Specific suitable GSTP1-specific primer sequences include SEQ ID NO: 13, SEQ ID NO: 15, SEQ ID NO: 17, SEQ ID NO: 19, SEQ ID NO: 21 and SEQ ID NO: 23.

The toxicity assessment methods of the invention next include a step of detecting GSTM1, GSTM3, GSTT1, and GSTP1 polymorphic alleles in the DNA obtained from the above-described PCR amplification steps. According to the invention, this detection step may first include combining the DNA obtained from the PCR amplification steps to detect GSTM1, GSTM3, GSTT1, and GSTP1 alleles. This saves time and cost by allowing detection methods to be performed on a single sample instead of on multiple samples. Multiple-sample detection may be performed within the scope of the invention if beneficial, however.

The detection step next generally includes conducting a gel electrophoresis of the combined DNA to allow detection of the individual alleles present using PCR product size differences and fluorescent tag differences. Alternatively, however, the step of detecting GSTM1, GSTM3, GSTT1 and GSTP1 polymorphic alleles in the DNA obtained from the PCR amplification steps may include conducting a capillary electrophoresis of the combined DNA and detection of the alleles present using PCR product size differences and fluorescent tag differences.

The toxicity assessment methods of the invention described above may be extended to allow gene dosage of GSTM1 alleles to be determined. More specifically, in the methods of the invention, the step of detecting GSTM1, GSTM3, GSTT1, and GSTP1 polymorphic alleles in the DNA obtained from the PCR amplification steps may be followed by a step of performing a long range PCR assay of a portion of the DNA to distinguish GSTM1*A/A or GSTM1*B/B homozygotes from GSTM1*A/null and GSTM1*B/null heterozygotes. The long range PCR assay of a portion of the DNA may be conducted using GSTM1*0-specific primer sequences. In some specific examples, the GSTM1*0-specific primer sequences may be SEQ ID NO: 27 and SEQ ID NO:28.

Similarly, the toxicity assessment methods of the invention may be extended to allow determination of GSTT1 gene dosage. In such extended methods, the step of detecting GSTM1, GSTM3, GSTT1 and GSTP1 polymorphic alleles in the DNA obtained from the PCR amplification steps may be followed by performing a long range PCR assay of a portion of the DNA to determine the gene dosage of GSTT1. The long range PCR assay of a portion of the DNA to determine the gene dosage of GSTT1 may be conducted using GSTT1*0-specific primer sequences. In some specific examples, the GSTT1*0-specific primer sequences may be SEQ ID NO: 33 and SEQ ID NO: 34. This step may alternatively be conducted using GSTT1*0-specific primer sequences and GSTT1/GSTT2-non-specific primer sequences. In some specific examples, the GSTT*O-specific primer sequences may be SEQ ID NO: 33 and SEQ ID NO: 34 and the GSTT1/GSTT2-non-specific primer sequences may be SEQ ID NO: 31 and SEQ ID NO: 32.

In a specific toxicity assessment assay of the invention, the step of performing PCR amplification of a portion of the DNA to detect GSTM1 alleles includes using primers having the sequences of SEQ ID NO: 1, SEQ ID NO: 3 and SEQ ID NO: 4; the step of performing PCR amplification of a portion of the DNA to detect GSTM3 and GSTT1 alleles includes using primers having the sequences of SEQ ID NO: 9, SEQ ID NO: 11, SEQ ID NO: 24 and SEQ ID NO: 25; and the step of performing PCR amplification of a portion of the DNA to detect GSTP1 polymorphisms includes using primers having the sequences of SEQ ID NO: 13, SEQ ID NO: 15, SEQ ID NO: 17, SEQ ID NO: 19, SEQ ID NO: 21 and SEQ ID NO: 23.

In alternate embodiments of the method of the invention, the steps of performing PCR amplification of a portion of the DNA are followed by the steps of identifying portions of the DNA which failed PCR amplification and performing single nucleotide extension verification assays to obtain the information originally sought by the PCR amplification. In this manner, a sample may be completely processed despite failure in the initial steps of the high throughput methods. These and other features and advantages of the present invention will become more fully apparent from the following description and appended claims, or may be learned by the practice of the invention as set forth hereinafter.

### BRIEF DESCRIPTION OF THE DRAWINGS

In order that the manner in which the above-recited and other features and advantages of the invention are obtained will be readily understood, a more particular description of the invention briefly described above will be rendered by reference to specific embodiments thereof which are illustrated in the appended drawings. Understanding that these drawings depict only typical embodiments of the invention and are not therefore to be considered to be limiting of its scope, the invention will be described and explained with additional specificity and detail through the use of the accompanying drawings in which:

FIG. 1 shows four electropherograms resulting from the high throughput genotyping method of the invention. Rows 1-4 represent DNA samples from four different subjects. Genotypes of each DNA sample were determined by the differential lengths of PCR products (increasing from left to right). Polymorphisms are represented from left to right in the following order: GSTP1-113T, GSTP1-113C, GSTM1*A, GSTM1*B, GSTM1*0 (absent allele), GSTP1-104A, GSTP1-104G, beta-actin (a control), GSTT1*1, GSTT1*0 (absent allele), GSTM3*B, and GSTM3*A;

FIG. 2 shows four electropherograms resulting from the single nucleotide extension assay. Single Nucleotide Extension (SNE) for GSTM1 and GSTP1 Single Nucleotide Polymorphisms (SNP). Rows 1-4 represent 4 SNE results from 4 different subjects. These are not the same subjects as for the samples in FIG. 1. The SNE primers are labeled at the bottom of the figure. The SNP results and their corresponding genotypes are given to the right of each row.

FIG. 3 shows the results of a long range PCR used to determine the gene dosage of GSTM1 and GSTT1. For the GSTM1 PCR, the presence of a 4.8 kB band indicates that at least one allele of GSTM1 is absent (e.g. GSTM1*null). For the GSTT1 PCR, the presence of a 1460 bp band indicates that at least one allele of GSTT1 is absent (e.g. GSTT1*0).

### DETAILED DESCRIPTION OF THE INVENTION

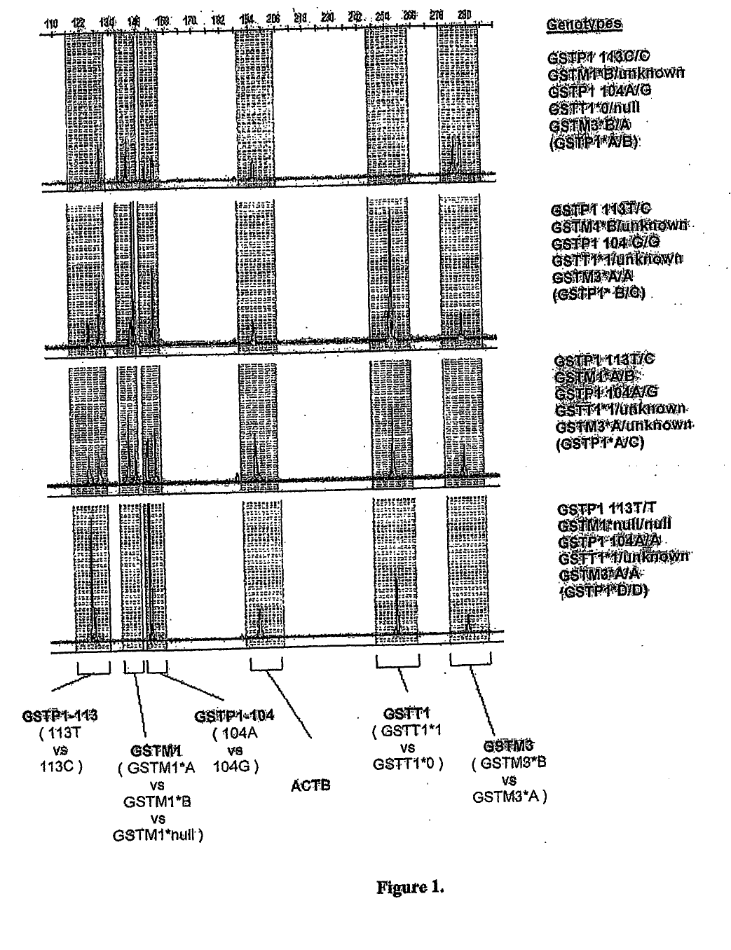

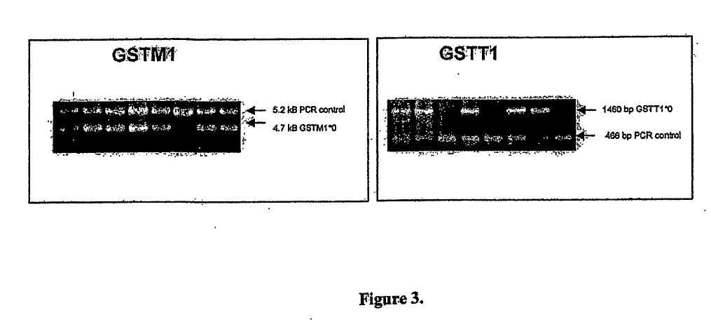

The presently preferred embodiments of the present invention may be best understood by reference to the drawings. It will be readily understood that the steps of the methods of the present invention, as generally described and illustrated herein, could be varied in many ways within the scope of the invention. Thus, the following more detailed description of the embodiments of the method of the present invention, as represented in FIGS. 1 through 3, is not intended to limit the scope of the invention, as claimed, but is merely representative of presently preferred embodiments of the invention.

An inexpensive high throughput assay for the detection of GST polymorphisms is disclosed herein which may be suitable for use with large clinical pharmacogenetic trials. A non-high throughput genotyping assay was also developed to determine the sensitivity and specificity of this high throughput assay, using single nucleotide extension (SNE). This SNE method allows the identification of polymorphic alleles for GSTM1 and GSTP1. Using these SNE assays, the overall sensitivity and specificity of the high throughput assay was demonstrated to be 92% and 97%, respectively.

The distribution of normal GST alleles for the state of Utah was evaluated using the methods within the scope of the invention and is presented herein. In addition, methods within the scope of the invention were used to evaluate cancer risk in patients carrying non-null alleles of GSTM1 and GSTT1. The high throughput assay disclosed herein was also used to demonstrate an increased risk of acute lymphoblastic leukemia, glial brain tumors, and osteosarcoma in patients carrying non-null alleles of GSTM1 and GSTT1.

Polymorphisms of glutathione S-transferase (GST) enzymes have been correlated with altered risk of many cancers, as well as altered response to and toxicity from cancer chemotherapy. Clinically-significant differences in cancer risk have been associated with polymorphic alleles of GSTM1, GSTM3, GSTP1, and GSTT1, whereas differences in response, toxicity, and outcome of treatment for cancer have been associated with polymorphic alleles of GSTM1, and GSTP1. Most of these results have been retrospective studies. As a result, prospective examinations of risk and response in the context of national trials are likely to lead to the discovery of new relationships between GST genotypes and treatment outcomes.

GST polymorphic alleles occur at prevalences that make them medically and socioeconomically important. The GST polymorphic alleles examined for this study occur in mixed populations at frequencies of at least 2.4-20% and as much as 40-84%. The new high throughput assay presented herein provides a low-cost, but highly accurate GST allele detection method that can be performed not only for sizeable study cohorts using peripheral blood but also for large control populations using readily available materials such as discarded Guthrie cards. The sensitivity (92-99%) of the assay of the invention with Guthrie cards suggests that the assay could be used with filter-based, room temperature stable buccal swab DNA collection methods, available commercially (Isocode®, Schleicher & Schuell, Keene, N.H.), which could be conveniently sent via mail without concern over therapy-related low blood counts. Other filter-based blood collection methods would also be usable in the scope of the invention. (Isocode®, Schleicher & Schuell, Keene, N.H.)

This high throughput assay has three advantages over existing high throughput assays. In contrast to other PCR-based GST assays, the protocol does not require restriction endonuclease digestion of the PCR products. In addition, unlike most other assays, the high throughput assay can reliably differentiate between the non-null GSTM1 alleles, GSTM1*A and GSTM1*B. Finally, the assay disclosed herein is the first comprehensive, high throughput method of assessing all four GST polymorphic alleles known to date to be clinically relevant. The only other reported high throughput PCR assay for GST genotypes, reported by Kristensen et al., does not distinguish between GSTM1*A and GSTM1*B. Kristensen, V. et al. Single tube multiplex polymerase chain reaction genotype analysis of STM1, GSTT1, and GSTP1: relationship of genotypes to TP53 tumor status and clinicopathological variables in breast cancer patients. Pharmacogenetics 8, 441-447 (1998). In addition, the assay of Kristensen et al. only determines GSTP1 polymorphisms at codon 104 of GSTP1, resulting in an incomplete and inconclusive GSTP1 genotype. Id.

A major appeal of the high throughput assay for detecting GST alleles of the invention is the low cost and time savings it offers over other methods. From a practical perspective, this methodology may determine the polymorphic alleles of all four different GST genes for 96 patient DNA samples within approximately 8 hours at a current cost of approximately $11.46 per sample. The assay has been implemented using capillary electrophoresis, which further speeds the assay without additional cost.

It is herein demonstrated that the assay of the invention has sample source versatility because DNA can be derived from a variety of readily available sources including peripheral blood, blood spotted onto filter paper such as Guthrie cards, expectorated mouthwash, and mouth swabs. For samples that fail the high throughput assay, the SNE verification assay can be performed at a cost of about $8.30 or $16.58 per single nucleotide polymorphism, depending upon whether all three or one single nucleotide polymorphism is assayed.

It is believed that the assay presented herein will expand the study of GSTs in human disease and will facilitate the incorporation of GST genotypes into the clinical management decision making. In a multivariate analysis of pediatric cancer patients that was limited by a small sample size, an increased risk of acute lymphoblastic leukemia, glial brain tumors, and osteosarcoma was demonstrated for patients carrying non-null alleles of GSTM1 and/or GSTT1. These results are consistent with a trend increase in GSTT1 non-null alleles among pediatric acute lymphoblastic leukemia patients in a Portuguese population. These results are initially counterintuitive since GSTM1 and GSTT1 are generally thought to be Phase II detoxifying enzymes responsible for the inactivation of carcinogens. However, GSTT1 is also known to have Phase I activity and the ability to activate carcinogens. Further, Bruning and co-workers have shown that GSTM1 and GSTT1 non-null alleles appear to increase the risk of trichloroethylene-induced renal cell carcinoma, which may be explained by the fact that GSTT1 is involved in the activation of some halogenated hydrocarbons rather than their inactivation. Bruning, T. et al., Influence of polymorphisms of GSTM1 and GSTT1 for risk of renal cell cancer in workers with long-term high occupational exposure to trichloroethene. Arch Toxicol 71, 596-599 (1997).

Previously-published reports examining GSTM1 and GSTT1 genotypes and acute lymphoblastic leukemia risk have been contradictory. GSTM1 and GSTT1 non-null alleles have either had no significant association with risk of acute lymphoblastic leukemia, or GSTT1 non-null alleles have been associated with reduced risk in general populations or population subsets. In contrast to many of these studies, the patient population studied herein is derived predominantly from a small founder population of Northern European descent, and the homogeneity of this population may allow for the statistical detection of small inherited variations in metabolism. At first glance, one might attribute differences in this and other studies to small sample sizes, ethnic heterogeneity, or the use of case versus population controls; however, the relative importance of GSTT1 polymorphisms may in fact depend on complex interactions between GSTT1 genotype, an individual's profile for many other polymorphic detoxification genes, and specific local environmental exposures. For these reasons, assessment of cancer risk by genotype may necessarily need to be restricted to specific geographic locations.

Pharmacogenetic applications of the assays of the invention may include (1) detection of individuals at risk for specific diseases followed by genetic counseling and prevention strategies, (2) tailored therapy for patients likely to have a GST-based altered response to therapy such as worsened response or increased toxicity, and (3) patient-specific utilization of allele-specific small-molecule inhibitors reversing chemotherapy resistance among cancers over-expressing certain GST polymorphic alleles.

The high throughput assays of the invention have been designed for use in large, prospective clinical trials. Ongoing studies are being conducted to determine how inheritance of common polymorphic alleles of the glutathione S-transferase detoxifying enzymes affects a child's susceptibility to common pediatric cancers, and to determine whether inheritance of unfavorable glutathione S-transferase alleles may serve as a predictor of a child cancer patient's response to chemotherapy. By studying GST genotypes in the context of large trials, it is hoped that patient populations may be identified that will benefit from tailored, pharmacogenetically-based preventative and therapeutic interventions.

### EXAMPLES

Patient Enrollment and Sample Acquisition

Informed consent was obtained from each subject or his/her guardian for the collection of DNA from peripheral blood samples, mouthwash, or buccal swabs. The informed consent was gathered based on a protocol approved by the University of Utah Institutional Review Board (IRB). Guthrie card samples used in experimentation (from which the patient identifiers had been removed) were obtained from the Utah Department of Health under the guidelines of the above-mentioned IRB-approved protocol.

Peripheral blood was obtained from some patients either through venipuncture or via an indwelling central venous catheter. Patients with white blood cell counts less than 1,000 cells per mm² were excluded from peripheral blood collection. As an alternative, children above the age of 5 years could participate in the studies by donating buccal epithelial cells. This was most commonly accomplished by vigorously swishing a commercial mouthwash (FreshBurst Listerine®, Warner Lambert, Morris Plains, N.J.) for 60 seconds and then expectorating into a collection container. Lum, A. & LeMarchand, L. A simple mouthwash method for obtaining genomic DNA in molecular epidemiological studies. Cancer Epidemiology, Biomarkers, and Prevention 7, 719-724 (1998). In children less than 5 years of age, buccal epithelial cells were obtained by swabbing the interior epithelium of their cheeks with four independent buccal brush strokes (CytoSoft Cytology Brushes, GentraSystems Inc., Minneapolis, Minn.).

In total, 109 peripheral blood samples, 14 mouthwash samples, 4 buccal swab samples, and 340 Guthrie Card samples were collected and analyzed for the determination of sensitivity and specificity. In the assays for the determination of pediatric cancer risk, 171 cancer patients ages 0-18 years were enrolled. Patients with low-grade gliomas and astrocytomas were collectively analyzed as glial brain tumors.

DNA Isolation

All biological samples obtained from consenting patients enrolled in the research were processed for genomic DNA by the Huntsman General Clinical Research Center at the University of Utah. DNA was isolated from the peripheral blood, buccal epithelial cells, or Guthrie cards contributed by using commercial protocols and reagents (PureGene™ Blood, PureGene™ Buccal Cell, and Generation™ Capture Card protocols, respectively) purchased from Gentra Systems, Minneapolis, Minn.

High Throughput Allele-Specific GSTM1 Detection

Following the isolation of genomic DNA from each sample, a PCR amplification step was conducted to allow the detection of GSTM1 alleles. Specifically, GSTM1 alleles were detected by fluorescent, allele-specific PCR using one forward primer, M1F (SEQ ID NO: 1) and two reverse primers, M1R-A (SEQ ID NO: 3) and M1R-B (SEQ ID NO: 4). The sequences of these GSTM1 PCR primer sequences are provided in Table 2. The sequences shown in Table 2 may include non-sequence-specific tails added to tagged primers to create PCR product length polymorphisms, or added to untagged primers to promote completion of non-templated nucleotide addition. Thus, SEQ ID NO: 1 (M1F) may be varied by conserving the base sequence SEQ ID NO: 2, while varying the bolded sequence. Further SEQ ID NO: 4 (M1R-B) may be varied by conserving the base sequence SEQ ID NO: 5 while varying the bolded sequence.

Polymorphic nucleotides were placed at the 3′ side of the reverse primers in order to achieve sequence specificity of PCR amplification. The beta-actin gene was co-amplified as a reaction control. Beta-actin forward and reverse primers sequences (SEQ ID NOS: 6, 7 and 8) are also presented in Table 2. SEQ ID NO: 8 represents a base reverse primer sequence which may be varied by the addition of a different bolded sequence than that of SEQ ID NO: 7. Reaction and thermal cycling conditions for the co-amplification reaction are presented in Table 3.

High Throughput Allele-Specific GSTM3 and GSTT1 Detection

GSTM3 and GSTT1 were co-amplified by PCR. GSTM3 alleles were detected directly as a size polymorphism. GSTT1 alleles were detected by the presence or absence of a PCR product. GSTM3 and GSTT1 primer sequences (SEQ ID NOS: 9, 10, 11 and 12; and SEQ ID NOS: 24, 25 and 26, respectively) and reaction/thermal cycling conditions are given in Tables 2 and 3, respectively.

High Throughput Allele-Specific GSTP1 Detection

Polymorphisms at codon 104 of GSTP1 were detected by fluorescent, allele-specific PCR. Polymorphic nucleotides were placed at the 3′ side of the forward primers in order to achieve sequence specificity of PCR amplification. Polymorphisms at codon 113 of GSTP1 were detected similarly. Primer sequences (SEQ ID NO: 13, 14, 15, 16, 17, 18, 19, 20, 21, 22 and 23) and reaction/thermal cycling conditions are given in Tables 2 and 3, respectively.

High Throughput Combined Detection of GSTM1, GSTM3, GSTT1, and GSTP1

Polymorphic Alleles

Through the design of product size differences and fluorescent tag differences for each of the PCR reactions mentioned above, all of the PCR reaction products could be combined and loaded into a single lane on an ABI PRISM® 373/377 Sequence Detection System (Applied Biosystems, Foster City, Calif.). Fragments were sized against the BRL 50-500 Tamra size standard (Invitrogen Corporation, Carlsbad, Calif.) using ABI GeneScan v3.1.2 software. Alleles were automatically scored using ABI Genotyper® v2.5 software based on size and color. Allele size and peak area information was captured and exported into Microsoft Excel® for peak area analysis. Peaks were scored only if the peak height was greater than 150 relative fluorescent units (RFU); otherwise, the peak was considered to be absent (GSTM1, GSTT1) or the reaction was considered to have failed (GSTM3, Beta-Actin, GSTP1). Samples in which the reaction failed could be tested using SNE methods as a backup. Specificity of GSTM1 and GSTP1 genotyping results was determined by comparing high throughput results to corresponding SNE assays that are described below.

Single Nucleotide Extension Allele-Specific GSTM1 Detection

To determine the specificity of the high throughput assay for non-null GSTM1 polymorphic alleles a single nucleotide extension (SNE) assay was used that employs the ABI Prism® SnaPshot™ ddNTP Primer Extension Kit (Applied Biosystems, Foster City, Calif.) to extend a primer one base pair at the single nucleotide polymorphism (or “SNP”) site. The first step of the SNE assay was to generate by PCR a specific 262 base pair (bp) fragment from genomic DNA that contains the GSTM1 SNP site. The nucleotide sequence of this PCR product has been conformed by sequencing. The second step of the SNE reaction was an extension (“mini-sequencing”) reaction performed on the PCR product using the SnaPshot™ kit. The SnaPshot™ chemistry allows a primer immediately 5′ of the SNP site to incorporate one dye-labeled ddNTP, thereby terminating the reaction after one nucleotide incorporation at the SNP site. Both sense and antisense extension reactions were performed as internal validation for the GSTM1 SNP result.

Primer sequences, reaction conditions, and thermal cycling conditions for the GSTM1 PCR reaction are given in Table 4. An aliquot of the PCR product from the first SNE reaction was monitored by electrophoresis on a 3% agarose gel. Subsequently, the non-electrophoresed PCR product was cleaned of free nucleotides and any single stranded DNA using ExoSAP-IT™ (USB Corporation, Cleveland, Ohio). The cleaned product of the GSTM1-specific PCR reaction was used as a template in a second SNaPshot™ reaction using a second set of sense and antisense primers that flank the SNP site. The unique dyes for terminator nucleotides A, C, G and T were dR6G, dTAMRA, dR110, and DROX, respectively. The forward and reverse primers used in the GSTM1 SNaPshot™ extension reaction yielded 43 base pair and 36 base pair products, respectively. Of note, the sense and antisense SNaPshot™ reactions for GSTP1-104 and GSTP1-113 were combined with the GSTM1 SNaPshot™ extension reaction for cost savings. After the SNaPshot™ extension reaction was complete, a second clean up was performed to remove all free dye-labeled ddNTPs. Products of both sense and antisense GSTM1 polymorphism single nucleotide extension were detected on an ABI PRISMS 373/377 Sequence Detection System (Applied Biosystems, Foster City, Calif.) in the same 5% polyacrylamide gel lane used for the GSTP1 104 and 113 SNE products, multiplexed by size and color. Results were scored for each sample only if sufficient DNA was present to successfully amplify both the GSTM1 SNE second reaction products (when present); otherwise, a single forward or reverse SNE result reaction was considered to be a failed genotype. A reaction for which an SNE product was not present when a high throughput GSTM1 product was present was also considered to have failed. Details for clean-ups and extension reactions used in the GSTM1 SNE assay are given in Table 4.

GSTP1 Codon 104 and Codon 113 Single Nucleotide Polymorphism Verification Assays

The principles of these SNE reactions are the same as described for the GSTM1 SNE reaction. In the first SNE reactions, the 57 base pair region surrounding the GSTP1-104 SNP or the 41 base pair region surrounding the GSTP1-113 SNP was amplified by PCR in separate reactions. Aliquots of PCR products were monitored by electrophoresis on a 3% agarose gel. Subsequently, the non-electrophoresed PCR product was cleaned of free nucleotides and any single stranded DNA using ExoSAP-IT™ (USB Corporation, Cleveland, Ohio).

As stated for the GSTM1 SNE reactions, the GSTP1-104 and GSTP1-113 specific PCR reaction products were used as templates in the subsequent SNaPshot™ extension reactions. The primers used for GSTP1-104 and GSTP1-113 extension reactions were the same as those used in the antecedent PCR reaction. As described above in the section for GSTM1 SNE assay, the GSTP1 extension reactions are multiplexed with the GSTM1 extension reaction and detected in the same electrophoresis gel lane. The size of the fragments produced in the final SNaPshot™ reaction for 104F, 104R, 113F, and 113R are 31 base pairs, 27 base pairs, 19 base pairs, and 23 base pairs respectively. Details for primer sequences, reaction conditions, and thermal cycling conditions used in the SNE assays are given in Table 4. Results for GSTP-104 and GSTP1-113 SNE products are scored as described earlier for the GSTM1 SNE assay.

GSTM1 Long Range PCR Assay for Determination of Gene Dosage

Neither the high throughput assay nor the single nucleotide extension assays was able to distinguish GSTM1*A or GSTM1*B homozygotes (i.e. GSTM1*A/A and GSTM1*B/B) from corresponding heterozygotes, GSTM1*A/null and GSTM1*B/null, respectively. Therefore, to determine gene dosage for GSTM1, a long range PCR to confirm or negate the presence of the GSTM1*0 allele was developed by modification of a previously reported protocol³⁴. Using the Expanded Long Template PCR System (Roche, Mannheim, Germany) and primers M2F10 (SEQ ID NO: 27) (5′AAGACAGAGGAAGGGTGCATTTGATA-3′) and M5R16A (SEQ ID NO: 28) (5′-ACAGACATTCATTCCCAAAGCGAC-3′), a 4.8 kilobase (B) product can be amplified in the presence of a GSTM1*0 allele. Specificity of this 4.8 kB product has been confirmed by sequencing. In this PCR reaction, a 5.2 kB product (or in some cases a polymorphic 5.4 kB product) of the human tissue plasminogen activator gene is co-amplified as a positive control using primers TPA7SF primer (SEQ ID NO: 29) (5′-GGAAGTACAGCTCAGAGTTCTG-3′) and the TPAREV primer (SEQ ID NO: 30) (5′-AGCGGGACGAATCCGATTTCAG-3′). The 25 μl reaction mixture contained 2-20 ng template DNA, 0.24 μM primers (TPA7SF (SEQ ID NO: 29), TPAREV (SEQ ID NO: 30), M5R16A (SEQ ID NO: 28) and M2F10 (SEQ ID NO: 27)), 1.9U DNA polymerase mix, 350 μM dNTPs, 2.75 mM MgCl₂, and 1× Roche Long Range Buffer System#2. After a 2 minutes incubation at 94° C., 10 cycles of 94° C. (10 seconds) were performed, followed by 67° C. (30 seconds) followed by 68° C. (5 minutes), then 15 cycles of 94° C. (15 seconds) followed by 67° C. (30 seconds plus an additional 5 seconds/cycle) followed by 68° C. (5 minutes), then 10 minutes at 68° C. PCR products were visualized on a 0.65% agarose gel stained with 0.5% ethidium bromide via ultraviolet light illumination.

GSTT1 Long Range PCR Assay for Determination of Gene Dosage

To determine gene dosage for GSTT1, a long range PCR was developed by modification of a previously reported protocol. Kerb, R et al. Influence of GSTT1 and GSTM1 Genotypes on Sunburn Sensitivity. Am J Pharmacogenomics 2, 147-54 (2002). Two sets of primers were used, one specific for the GSTT1*0 allele and the other non-specific for the GSTT1/GSTT2 alleles. The latter set served as an internal control for the PCR reaction.

The GSTT1*A-F (SEQ ID NO: 31) and GSTT1*A-R (SEQ ID NO: 32) primers with sequences 5′-AATGCTTTGTGGACTGCTGAGG-3′ and 5′-TGATGCATGTGAGTGCTGTGG-3′, respectively, generated a 455 bp product in the presence of GSTT1*1 or GSTT2 alleles. The primers GSTT1*0-F (SEQ ID NO: 33) and is GSTT1*0-R (SEQ ID NO: 34), have sequences 5′-TACAGTTGTGAGCCACCGTACC-3′ and 5′-ATAGTTGCTGGCCCCCTCATT-3′, yielded a 1460 bp product when the GSTT1*0 allele was present. Specificity of this 1460 bp product has been confirmed by nucleotide sequencing. The PCR mixture contained 0.01 μM GSTT1*A-F (SEQ ID NO: 31), 0.01 μM GSTT1*A-R (SEQ ID NO: 32), 0.02 μM GSTT*0-F (SEQ ID NO: 33), 0.0211M GSTT*0-R (SEQ ID NO: 34), 0.25U Taq Platinum Polymerase (Invitrogen Corporation, Carlsbad, Calif.), 0.2 nM spermidine HCl, 1.5 mM MgCl₂, 40 mM NaCl, 10 mM Tris-HCl (pH 8.3), 200 μM dNTPs each, and 9% DMSO. Reaction volume was 20 μL. After a 5 minutes incubation at 94° C., 6 cycles of 94° C. (30 seconds) were performed, followed by 68° C. (30 seconds with decrement of 1° C. per cycle) followed by 72° C. (2 minutes), then 30 cycles of 94° C. (30 seconds) followed by 63° C. (30 seconds) followed by 72° C. (2 minutes), then 10 minutes at 72° C. The resulting PCR products were visualized by electrophoresis on a 1% agarose gel stained with 0.5% ethidium bromide via ultraviolet light illumination.

Statistical Analysis

Disease risk was analyzed univariately by comparing disease-free patients (controls) vs. a specific group of patients with respect to a particular genotype. The controls were not matched to the patients. Chi-square test was used to detect an association between genotype and disease. Whenever the cell counts were small, an exact test was used. No adjustment for multicomparisons has been made. Multivariate analysis was performed with a smaller subset of the data. The presence of a particular disease was used as a binary response variable, and logistic regression was used for the analysis. The set of explanatory variables was constructed using factorial coding of various genotypes. Backward variable selection procedures and a likelihood ratio test were used to select significant variables.

High Throughput Assay for Detection of GSTM1, GSTM3, GSTP 1, and GSTT1 Polymorphic Alleles

Four representative electropherograms for the high throughput assay are shown in FIG. 1. Differentiation of the alleles was based on size and fluorescent tag color. Potential peaks read from left to right correspond to the following polymorphisms or control genes: GSTP1-113T, GSTP1-113C, GSTM1*A, GSTM1*B, GSTM1*0 (absent allele), GSTP1-104A, GSTP1-104G, beta-actin (a control), GSTT1*1, GSTT1*0 (absent allele), GSTM3*B, and GSTM3*A. As shown in Table 1, the GSTP1 genotype is determined from the combined detections of single nucleotide polymorphisms at the GSTP1-104 and GSTP1-113 codons.

The sensitivity and specificity of the high throughput assay are presented in Table 5. Sensitivity has been defined as the proportion of successful reactions for each polymorphic GST gene, whereas specificity (for GSTM1 and GSTP1) was defined as the level of concordance with the results of an SNE verification assay. For GSTM3 and GSTT1 specificity was not measured because the length polymorphisms of GSTM3 and the presence/absence polymorphisms of GSTT1 are less prone to errors of specificity than the single nucleotide polymorphisms of GSTM1 and GSTP1. Sensitivity and specificity for high throughput and SNE assays using mouthwash and buccal swab derived samples were not calculated as percentages because of the small numbers of samples.

Overall, the sensitivity and specificity of the high throughput assay were 85-99% and 93-100%, respectively, depending on the source of genomic DNA. The high throughput assay was more sensitive for Guthrie card derived DNA samples (sensitivity 92-99%) than for peripheral blood derived samples (85-89%). Without being limited to any one theory, it is thought that Guthrie card-derived DNA may be more fragmented than peripheral blood-derived DNA, and therefore may present an advantage for PCR amplification so long as the template regions are relatively short. Of note, peripheral blood-derived DNA sample results were slightly more specific (98-100%) than Guthrie card derived samples (93-100%)

Verification Assays Using Single Nucleotide Extension

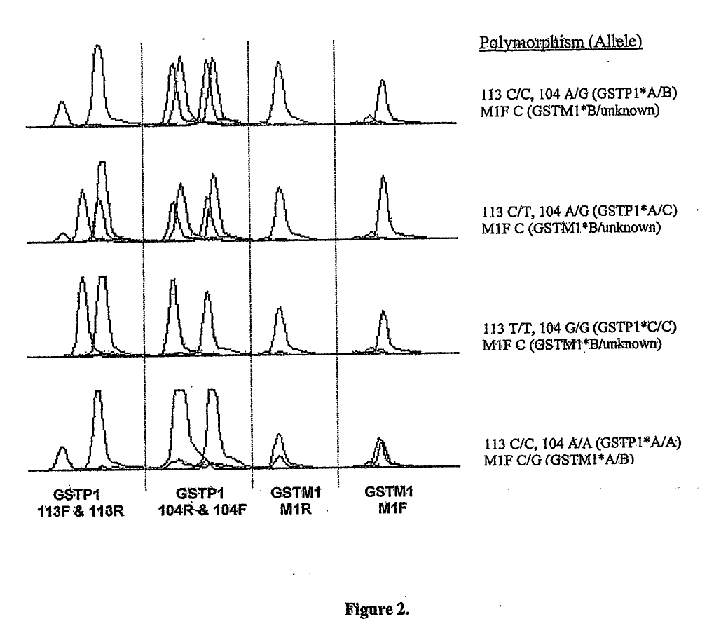

Four representative electropherograms for the SNE assay are presented in FIG. 2. Single nucleotide polymorphisms are discriminated both by size and fluorescent tag. From left to right the peaks correspond to the following SNE primers: GSTP1-113F (SEQ ID NO: 45), GSTP1-113R (SEQ ID NO: 46, SEQ ID NO: 47 (without tail)), GSTP11-104R (SEQ ID NO: 43, SEQ ID NO: 44 (without tail)), GSTP1-104F (SEQ ID NO: 41, SEQ ID NO: 42 (without tail)), GSTM1-M1R, (SEQ ID NOS: 3, 4) and GSTM1-M1F (SEQ ID NO: 1). Results are determined from the color of each peak, as described in the figure legend.

The sensitivity and specificity of the combined SNE assay were 90-98% and 100%, respectively, depending on the source of genomic DNA. Unlike the high throughput assay, sensitivity was significantly better for peripheral blood-derived DNA samples (98%) than for Guthrie card-derived DNA samples (90-98%). This degree of sensitivity of the SNE assay presents an opportunity to successfully genotype peripheral blood-derived DNA samples that have failed the high throughput assay, albeit at a higher per sample cost. Nevertheless, the combination of a first-line high throughput assay and a second line SNE assay provides a cost-effective means of GST genotyping large cohorts from peripheral blood at high levels of sensitivity and specificity.

Long Range PCR Assays for Determination of GSTM1 and GSTT1 Gene Dosage

Most clinical studies of GSTM1 and GSTT1 polymorphisms to date have been disadvantaged by the inability to differentiate homozygous non-null GSTM1 or GSTT1 genotypes from heterozygous non-null plus null genotypes. To overcome this problem, long range PCR assays to determine whether a GSTM1*0 or GSTT1*0 allele was present, respectively, were developed to calibrate gene dosage determination for the high throughput assay. It was originally expected that the gene dosage of GSTM1 and GSTT1 may be determined by examining the area under the curve (AUC) ratios for GSTM1:Beta-actin and GSTT1:GSTM3 in the high throughput assay. The long range PCR assays for GSTM1 and GSTT1 were developed to provide a gold standard for the true gene dosage of GSTM1 and GSTT1. Although high throughput assay AUC ratios corresponded to true gene dosage, AUC ratios did not provide a clear cutoff to distinguish between the presences of one versus two non-null alleles for either gene. Nevertheless, the long range assays described herein are robust and can be used to supplement the high throughput assay in the determination of GSTM1 and GSTT1 gene dosage.

GST Genotype and Allele Frequencies in the State of Utah

The availability of Guthrie card samples from which patient identifiers have been removed provides an ideal, unbiased cross-sectional resource for the determination of GST polymorphic allele frequencies. Table 6 shows the frequency of the different GST polymorphic alleles from a random sampling of the population in the state of Utah determined from 340 Guthrie cards of infants born in the year 2001-2002. These allele frequencies differ interestingly from those previously reported for Caucasian population of British/Northern European descent. Using Ordinal Logistic Regression, the GSTM1, GSTM3, and GSTT1 allele frequencies are comparable to Northern European populations, whereas GSTP1*B and GSTP1*C frequencies are significantly higher and GSTP1*A frequencies are significantly lower (p=0.034). Most striking is a three-fold higher frequency of the GSTP1*C allele (7.5 versus 2.4%), the highest frequency of this allele in any population reported to date. The over-representation of the GSTP1*C allele may be, in part, due to the fact that Utah is an out bred population, founded by a small cohort of Northern European descendents.

GST Genotype and Risk of Pediatric Cancers

The GST polymorphic allele frequencies for pediatric cancer patients are shown in Table 7. Surprisingly, only GSTM1 and GSTT1 polymorphic alleles achieved significance by multivariate analysis in this study (Table 8). In each case individuals with two null alleles of each gene were associated with the lower relative risk. Specifically, a 4.3-fold increased relative risk of acute lymphoblastic leukemia was found among subjects that carry one non-null (GSTM1*A or GSTM1*B) allele of GSTM1 over individuals with two null alleles (95% confidence intervals 2.581-12.415 (p=0.001) and 1.8-10.2 (p<0.001), respectively). The relative risk of acute lymphoblastic leukemia was also found to be 2.6-fold higher among individuals with one non-null allele of GSTT1 as compared to individuals with two null alleles (95% confidence intervals 1.1-6.3, p=0.035). Similar outcomes were found among glial brain tumors and osteosarcoma. The relative risk of glial brain tumors was 4.9-fold higher for subjects carrying one non-null (GSTM1*A) allele of GSTM1 in comparison to subjects with two null alleles (95% confidence intervals 1.5-16, P=0.009). For osteosarcoma the presence of GSTM1*A or GSTM1*B non-null alleles resulted in 6.9-16-fold increased relative risk over genotypes with GSTM1*0 null alleles (confidence intervals 1.1-42.7 (p=0.038) and 2.8-92.2 (p=0.002), respectively).

TABLE 1
Polymorphic Alleles of Glutathione S-Transferase Genes

| Class/ | | Allelic | | |
|---|---|---|---|---|
| Subclass | Locus | Variants | Codon | Significance |
| Mu | | | | |
| GSTM1 | 1p13.3 | GSTM1*0 | | Absent allele |
| | | GSTM1*A | Lys¹⁷³ (AAG ) | Altered hinge between two alpha |
| | | GSTM1*B | Asn¹⁷³ (AAC ) | helixes at a dimerization site |
| GSTM3 | 1p13.3 | GSTM3*A | Full Intron 6 | Unknown; The deletion generates a YY1 |
| | | GSTM3*B | 3 bp Deletion | repressive transcription factor |
| | | | in Intron 6 | recognition site |
| Pi | | | | |
| GSTP1 | 11q13 | GSTP1*A | Ile¹⁰⁴ (A TC), | Modified contacts at the |
| | | | Ala¹¹³ (GC G) | binding site for electrophilic |
| | | GSTP1*B | Val¹⁰⁴ (G TC), | carcinogens (H-site) |
| | | | Ala¹¹³ (GC G) | |
| | | GSTP1*C | Val¹⁰⁴ (G TC), | |
| | | | Val¹¹³ (GT G) | |
| | | GSTP1*D^{†} | Ile¹⁰⁴ (A TC), | |
| | | | Val¹¹³ (GT G) | |
| Theta | | | | |
| GSTT1 | 22q11 | GSTT1*0 | | Absent Allele |
| | | GSTT1*1 | | |
| Single nucleotide polymorphisms are underlined. | | | | |
| ^{†}GSTP1*D is an extremely rare allele demonstrated in case reports by PCR, but for which a cDNA or genomic DNA is yet to be isolated. | | | | |
| References: | | | | |
| GSTM1: Fryer, A., Zhao, L., Alldersea, J., Pearson, W. R. & Strange, R. C. Use of site-directed mutagenesis of allele-specific PCR primers to identify the GSTM1A, GSTM1B, GSTM1A,B and GSTM1 null polymorphisms at the glutathione S-transferase, GSTM1 locus. Biochemical Journal 295, 313-315 (1993). | | | | |
| GSTM3: Krajinovic, M., Labuda, D., Richer, C., Karimi, S. & Sinnett, D. Susceptibility to Childhood Acute Lymphoblastic Leukemia: Influence of CYP1A1, CYP2D6, GSTM1, and GSTT1 Genetic Polymorphisms. Blood 93, 1496-1501 (1999); Inskip, A. et al. Identification of polymorphism at the glutathione S-transferase, GSTM3 locus: evidence for linkage with GSTM1*A. Biochemical Journal 312, 713-716 (1995). | | | | |
| GSTP1: Ali-Osman, F., Akande, O., Antoun, G., Mao, J.-X. & Buolamwini, J. Molecular Cloning, Characterization, and Expression in Escherichia coli of Full-length cDNAs of Three Human glutathione S-Transferase Pi Gene Variants. The Journal of Biological Chemistry 272, 10004,-10012 (1997); Watson, M. A., Stewart, R. K., Smith, G. B. J., Massey, T. E & Bell, D. A. Human glutathione S-transferase P1 polymorphisms: relationship to lung tissue enzyme | | | | |
| # activity and population frequency distribution. Carcinogenesis 19, 275-280 (1998). | | | | |

GSTT1: Pemble, S. et al. Human glutathione S-transferase Theta (GSTT1): cDNA cloning and characterization of a genetic polymorphism. Biochemical Journal 300, 271-276 (1994).

TABLE 2
PCR Primers for High Throughput Genotyping

| | | | |
|---|---|---|---|
| Gene | Primer Name | Fwd/Rvs | PCR Product(s) |

| | | | |
|---|---|---|---|
| GSTM1 | M1F | Fwd | 0 bp (GSTM1*0) |
| | SEQ ID NO: 1 | 5′-GTTTCTTCTGCTTCACGTGTTAT | |
| | | GAAGGTTC-3′ | |
| | SEQ ID NO: 2 | 5′-5′-CTGCTTCACGTGTTATGAAG | |
| | | GTTC-3′ | |
| | M1R-A | Rvs | 142 bp(GSTM1*A) |
| | SEQ ID NO: 3: | 5′-(TET)TTGGGAAGGCGTCCAAGC | |
| | | AC-3′ | |
| | M1R-B | Rvs | 145 bp(GSTM1*B) |
| | SEQ ID NO: 5: | 5′-(FAM)TCTTTGGGAAGGCGTCCA | |
| | | AGCAG-3′ | |
| | SEQ ID NO: 6: | 5′-TTGGGAAGGCGTCCAAGCA-3′ | |
| Beta-Actin | ACTB-A | Fwd | 194 bp (control) |
| | Sequence: | 5′-CCTCCCTGGAGAAGAGTAC-3′ | |
| | AGTB-B | SEQ ID NO: 7, 8 | Rev |
| | SEQ ID NO: 7: | 5′-(FAM)GTTTCTGTGTTGGCGTA | |
| | | CAGGTCTTT-3′ | |
| | SEQ ID NO: 8: | 5′-GTGTTGGCGTACAGGTCTTT-3′ | |
| GSTM3 | M3F | Fwd | 287 bp (GSTM3*A) |
| | SEQ ID NO: 9: | 5′-(FAM)GTTTCTCCTCAGTACTTG | |
| | | GAAGAGCT-3′ | |
| | SEQ ID NO: 10: | 5′-CCTCAGTACTTGGAAGAGCT-3′ | |
| | M3R | Rvs | 284 bp (GSTM3*B) |
| | SEQ ID NO: 11: | 5′-GTTTCTCACATGAAAGCCTTCAG | |
| | | GTT-3′ | |
| | SEQ ID NO: 12: | 5′-CACATGAAAGCCTTCAGGTT-3′ | |
| GSTP1 | P1-104FA | Fwd | 150 bp (Ile¹⁰⁴) |
| | SEQ ID NO: 13: | 5′-(FAM)GTTTCTGACCTCCGCTG | |
| | | CAAATACA-3′ | |
| | SEQ ID NO: 14: | 5′-GACCTCCGCTGCAAATACA-3′ | |
| | P1-104FG | Fwd | 153 bp (Val¹⁰⁴) |
| | SEQ ID NO: 15: | 5′-(TET)GTTTCTCTTGACCTCCG | |
| | | CTGCAAATACG-3′ | |
| | SEQ ID NO: 16: | 5′-GACCTCCGCTGCAAATACG-3′ | |
| | P1-104R | Rvs | |
| | SEQ ID NO: 17: | 5′-GTTTCTCAGCCCAAGCCACCTG | |
| | | A-3′ | |
| | SEQ ID NO: 18: | 5′-TCAGCCCAAGCCACCTGA-3′ | |
| | P1-113FT | Fwd | 130 bp (Ala¹¹³) |
| | SEQ ID NO: 19: | 5′-(TET)CTTTGGTGTCTGGCAGG | |
| | | AGGT-3′ | |
| | SEQ ID NO: 20: | 5′-GGTGTCTGGCAGGAGGT-3′ | |
| | P1-113FC | Fwd | 126bp(Val¹¹³) |
| | SEQ ID NO: 21: | 5′-(FAM)GGTGTCTGGCAGGAGG | |
| | | C-3′ | |
| | SEQ ID NO: 22: | 5′- | |
| | P1-113R | Rvs | |
| | SEQ ID NO: 23: | 5′-GTTTCTTGGTCTCCCACAATGA | |
| | | AGG-3′ | |
| GSTT1 | T1F | Fwd | 255 bp (GSTT1) |
| | SEQ ID NO: 24: | 5′-(FAM)TTCCTTACTGGTCCTCA | |
| | | CATCTC-3′ | |
| | T1R | Rvs | 0 bp(GSTT1*0) |
| | SEQ ID NO: 25: | 5′-GTTTCTACAGACTGGGGATGGA | |
| | | TGGTT-3′ | |
| | SEQ ID NO: 26: | 5′-TACAGACTGGGGATGGATGGT | |
| | | T-3′ | |

Legend:
Fwd = forward primer, Rvs = reverse primer.
TET and FAM are fluorescent tags used to detect sequences by automated polacrylamide gel electrophoresis.
On tagged primers bolded nucleotides indicate non-sequence specific tails used to create PCR product length polymorphisms.
On untagged primers bolded nucleotides indicate non-sequence specific tails added to promote completion of non-templated nucleotide addition^{39,40}.
Underlined nucleotides indicate single nucleotide polymorphisms.

TABLE 3
High Throughput Genotyping: Reaction and Thermal Cycling Conditions

| Reaction | Reaction Conditions | Thermal Cycling Conditions^{†} |
|---|---|---|
| GSTM1 | ACTB-A 0.50 μM, ACTB-B 0.50 μM, M1F | 94° C. (5 minutes), 30 cycles |
| | (SEQ ID NO: 1) 0.5 μM, M1RA (SEQ ID | of 94° C. (20 seconds) |
| | NO: 3) 0.25 μM, M1RB (SEQ ID NO: 4) | followed by 52° C. (20 |
| | 0.25 μM. | seconds) followed by 72° C. |
| | Taq Platinum Polymerase 0.25U (Invitrogen | (40 seconds), then 72° C. (10 |
| | Corporation, Carlsbad, CA), Spermidine | minutes). |
| | HCL 0.2 mM, MgCl₂ 1.5 mM, | |
| | NaCl 40 mM, Tris-HCL (pH 8.3) 10 mM, | |
| | deoxynucleotide triphosphates 200 μM each. | |
| | Reaction volume 20 μL. | |
| GSTM3/ | T1F (SEQ ID NO: 24) 0.5 μM, T1R (SEQ ID | 94° C. (5 minutes), 25 cycles |
| GSTT1 | NO: 25) 0.5 μM, M3F (SEQ ID NO: 9) 0.5 | of 94° C. (20 seconds) |
| | μM, M3R (SEQ ID NO: 11) 0.5 μM. All | followed by 58° C. (20 |
| | other conditions are the same as for GSTM1. | seconds) followed by 72° C. |
| | | (40 seconds), then 72° C. (10 |
| | | minutes). |
| GSTP1-104 | P1-104FA (SEQ ID NO: 13) 0.25 μM, P1- | 94° C. (5 minutes), 25 cycles |
| | 104FG (SEQ ID NO: 15) 0.125 μM, P1-104R | of 94° C. (20 seconds) |
| | (SEQ ID NO: 17) 0.5 μM. All other | followed by 64° C. (20 |
| | conditions are the same as for GSTM1. | seconds) followed by 72° C. |
| | | (40 seconds), then 72° C. (10 |
| | | minutes). |
| GSTP1-113 | P1-113FC (SEQ ID NO: 21) 0.25 μM, P1- | Cycling conditions are the |
| | 113FT (SEQ ID NO: 19) 0.125 μM, P1-113R | same as for GSTP1-104. |
| | (SEQ ID NO: 23) 0.5 μM. All other | |
| | conditions are the same as for GSTM1. | |

^{†}Samples were amplified in an MJ Research PTC250 thermal cycler (MJ Research, Inc., Watertown, MA).

TABLE 4
Reaction and Thermal Cycling Conditions for Single Nucleotide Extension Assays

| | |
|---|---|
| GSTM1 | PRIMERS |
| | First Reaction: |
| | M1nest2-F: (SEQ ID NO: 35) 5′-TGCTTCACGTGTTATGGAGG- 3′ |
| | M1nest2-R: (SEQ ID NO: 36) 5′-CATGCGAGTTATTCTGTGTGTAGC-3′ |
| | Second Reaction: |
| | SNEM1-F: (SEQ ID NO: 37, SEQ D NO: 38 (without tail)) |
| | 5′-GTTTCTGTTTCTGTTTCTGTTTCACCGTATATTTGAGCCCAA-3′ |
| | SNEM1-R: (SEQ ID NO: 39, SEQ ID NO: 40 (without tail)) |
| | 5′-GTTTCTGTTTCTGTTTCTGGGAAGGCGTCCAAGCA-3′ |
| | REACTION CONDITIONS |
| | First Reaction: |
| | NEST2-F (SEQ ID NO: 35) 0.50 μM, NEST2-R (SEQ ID NO: 36) 0.50 μM. |
| | Taq Platinum Polymerase 0.25U (Invitrogen Corporation, Carlsbad, CA), |
| | spermidine HCl 0.2 mM, MgCl₂ 1.5 mM, NaCl 40mM, Tris-HCl (pH 8.3) 10 mM, |
| | dNTPs 200 μM each. Reaction volume 20 μL. |
| | First Clean-up: |
| | PCR product 5 μl, ExoSAP-IT ™ SAP (USB Corporation, Cleveland, OH) 2 μL. |
| | Second Reaction: |
| | GSTM1, GSTP1-104 (SEQ ID NO: 13, 15, 17) and GSTP1-113 (SEQ ID NO: 19, |
| | 21, 23) cleaned PCR products 0.15 pmoles each air dried in the same reaction tube, |
| | SNaPshot ™ Ready Reaction Premix (Applied Biosystems, Foster City, CA) 5 μl, |
| | single nucleotide extension primers (SNEM1-F (SEQ ID NO: 37), SNFM1-R |
| | (SEQ ID NO: 38), SNEP1-104F (SEQ ID NO; 13, 15), SNEP1-104R (SEQ ID |
| | NO: 17), SNEP1-113F (SEQ ID NO: 19, 21), and SNEP1-113R (SEQ ID NO: |
| | 23)) 0.50 μM each. Reaction volume 10 μL. |
| | Second Clean-up: |
| | SAP (USB Corporation, Cleveland, OH) 2 μl, SNaPshot ™ extension reaction 10 |
| | μl. |
| | THERMAL CYCLING CONDITIONS |
| | First Reaction: |
| | 94° C. (5 minutes), 8 cycles of 94° C. (20 seconds) followed by 68° C. (20 seconds) |
| | minus 1° C. per cycle followed by 72° C. (40 seconds), 30 cycles of 94° C. (20 |
| | seconds) followed by 60° C. (20 seconds) followed by 72° C. (40 seconds), then |
| | 72° C. (10 minutes). |
| | First Clean-up: |
| | 37° C. (30 minutes) followed then 80° C. (15 minutes). |
| | Second Reaction: |
| | 25 cycles of 94° C. (10 seconds), 50° C. (5 seconds), then 60° C. (30 seconds). |
| | Second Clean-up:. |
| | 37° C. (60 minutes), then 72° C. (15 minutes). |
| GSTP1 | PRIMERS |
| Codon | 104F: (SEQ ID NO: 41, SEQ ID NO: 42 (without tail)) 5′- |
| 104 | GTTTCTGTTTCGGACCTCCGCTGCAAATAC-3′ |
| or | 104R: (SEQ ID NO: 43, SEQ ID NO: 44 (without tail)) 5′- |
| | GTTTCTGTTGTTGTAGATGAGGGAGA-3′ |
| GSTP1 | 113F: (SEQ ID NO: 45) 5′-GTGGTGTCTGGCAGGAGG- 3′ |
| Codon | 113R: (SEQ ID NO: 46, SEQ ID NO: 47 (without tail)) 5′- |
| 113 | TCTCACATAGTCATCCTTGCCC- 3′ |
| | REACTION CONDITIONS |
| | First Reaction: |
| | 104F or 113F 0.50 μM, 104R or 113R 0.50 μM. |
| | All other conditions are the same as for the first reaction for GSTM1. |
| | First Clean-up: |
| | Same as for GSTM1 |
| | Second Reaction: |
| | Same as for GSTM1 |
| | Second Clean-up: |
| | Same as for GSTM1 |
| | THERMAL CYCLING CONDITIONS |
| | First Reaction: |
| | 94° C. (5 minutes), 4 cycles of 94° C. (20 seconds) followed by 68° C. (20 seconds) |
| | minus 1° C. per cycle followed by 72° C. (40 seconds), 30 cycles of 94° C. (20 |
| | seconds) followed by 64° C. (20 seconds) followed by 72° C. (40 seconds), then |
| | 72° C. (10 minutes). |
| | First Clean-up: |
| | Same as for GSTM1 |
| | Second Reaction: |
| | Same as for,GSTM1 |
| | Second Clean-up: |
| | Same as for GSTM1 |

Bolded nucleotides represent non-sequence specific tails added to adjust the product length to allow for multiplexing of the final SNaPshot ™ fragments to be run in a signal gel lane. Samples were amplified in an MJ Research PTC250 thermal cycler (MJ Research, Inc., Watertown, MA).

TABLE 5
Sensitivity and Specificity of GST Genotyping

| | BLOOD | BLOOD | Guthrie Card | Guthrie Card | Mouthwash | Mouthwash | Buccal Swab | Buccal Swab |
|---|---|---|---|---|---|---|---|---|
| | Sensitivity | Specificity | Sensitivity | Specificity | Sensitivity | Specificity | Sensitivity | Specificity |
| M1 by High | 93 of 109 (85%) | 54 of 55 (98%) | 336 of 340 (99%) | 76 of 82 (93%) | 12 of 14 | 11 of 11 | 4 of 4 | 2 of 2 |
| Throughput | | | | | | | | |
| M1 by SNE | 65 of 66 (98%) | 65 of 65 (100%) | 80 of 82 (98%) | 80 of 80 (100%) | 12 of 13 | 12 of 12 | 2 of 2 | 2 of 2 |
| M3/T1 by | 94 of 109 (86%) | | 309 of 274 (91%) | | 10 of 13 | | 3 of 4 | |
| High | | | | | | | | |
| Throughput | | | | | | | | |
| P1-104 by | 97 of 109 (89%) | 59 of 59 (100%) | 336 of 340 (99%) | 151 of 152 (99%) | 11 of 14 | 11 of 11 | 4 of 4 | 2 of 2 |
| High | | | | | | | | |
| Throughput | | | | | | | | |
| P1-104 by | 65 of 66 (98%) | 65 of 65 (100%) | 146 of 163 (90%) | 146 of 146 (100%) | 12 of 13 | 12 of 12 | 2 of 2 | 2 of 2 |
| SNE | | | | | | | | |
| P1-113 by | 97 of 109 (89%) | 58 of 58 (100%) | 313 of 340 (92%) | 152 of 155 (98%) | 11 of 14 | 11 of 11 | 4 of 4 | 2 of 2 |
| High | | | | | | | | |
| Throughput | | | | | | | | |
| P1-113 by | 65 of 66 (98%) | 65 of 65 (100%) | 156 of 164 (95%) | 156 of 156 (100%) | 12 of 12 | 12 of 12 | 2 of 2 | 2 of 2 |
| SNE | | | | | | | | |

TABLE 6
GST Genotype Frequencies for the State of Utah
determined using Guthrie Cards

| | | |
|---|---|---|
| (Genotype) | (n) | (Percent) |

GSTM1 Genotype Frequencies

| | | |
|---|---|---|
| GSTM1*0/0 | 183 | 56 |
| GSTM1*A/unknown | 82 | 25 |
| GSTM1*A/B | 16 | 5 |
| GSTM1*B/unknown | 45 | 14 |
| TOTAL | 326 | 100 |

GSTM3 Genotype Frequencies

| | | |
|---|---|---|
| GSTM3*A/A | 224 | 75 |
| GSTM3*A/B | 71 | 24 |
| GSTM3*B/B | 5 | 2 |
| TOTAL | 300 | 100 |

GSTT1 Genotype Frequencies

| | | |
|---|---|---|
| GSTT1*0/0 | 66 | 22 |
| GSTT1*1/unknown | 234 | 78 |
| TOTAL | 300 | 100 |

GSTP1 Genotype Frequencies

| | | |
|---|---|---|
| GSTP1*A/A | 122 | 42 |
| GSTP1*A/B | 102 | 35 |
| GSTP1*A/C | 30 | 10 |
| GSTP1*B/B | 22 | 8 |
| GSTP1*B/C | 11 | 4 |
| GSTP1*C/C | 1 | 0 |
| GSTP1*C/D | 0 | 0 |
| TOTAL | 288 | 100 |

GSTP1 Allele Frequencies

| | | |
|---|---|---|
| GSTP1*A | 376 | 65 |
| GSTP1*B | 157 | 27 |
| GSTP1*C | 43 | 7.5 |
| GSTP1*D | 0 | 0 |
| TOTAL | 576 | 100 |

TABLE 7
GST Polymorphism Frequencies among Pediatric Cancer Patients

| | | | | | | | Ewing's | | Gllal Brain | |
|---|---|---|---|---|---|---|---|---|---|---|
| | All Utah | | All Patients | | ALL | | Sarcoma | | Tumors | |
| | (n) | (%) | (n) | (%) | (n) | (%) | (n) | (%) | (n) | (%) |
| GSTM1 Genotypes | | | | | | | | | | |
| GSTM1*0/0 | 183 | 56 | 93 | 50 | 48 | 51 | 6 | 46 | 8 | 47 |
| GSTM1*A/unknown | 82 | 25 | 58 | 31 | 30 | 32 | 4 | 31 | 8 | 47 |
| GSTM1*A/B | 16 | 5 | 5 | 3 | 2 | 2 | 2 | 15 | 0 | 0 |
| GSTM1*B/unknown | 45 | 14 | 33 | 16 | 14 | 15 | 1 | 8 | 1 | 6 |
| TOTAL | 326 | 100 | 189 | 100 | 94 | 100 | 13 | 100 | 17 | 100 |
| GSTM3 Genotypes | | | | | | | | | | |
| GSTM3*A/A | 224 | 75 | 110 | 67 | 53 | 62 | 9 | 75 | 11 | 85 |
| GSTM3*A/B | 71 | 24 | 52 | 32 | 30 | 35 | 3 | 25 | 2 | 15 |
| GSTM3*B/B | 5 | 2 | 2 | 1 | 2 | 23 | 0 | 0 | 0 | 0 |
| TOTAL | 300 | 100 | 164 | 100 | 85 | 100 | 12 | 100 | 13 | 100 |
| GSTT1 Genotypes | | | | | | | | | | |
| GSTT1*0/0 | 66 | 22 | 24 | 15 | 9 | 11 | 3 | 25 | 1 | 7 |
| GSTT1*1/unknown | 234 | 78 | 141 | 85 | 72 | 89 | 9 | 75 | 14 | 93 |
| TOTAL | 300 | 100 | 165 | 100 | 81 | 100 | 12 | 100 | 15 | 100 |
| GSTP1 Genotypes | | | | | | | | | | |
| GSTP1*A/A | 122 | 42 | 72 | 42 | 37 | 45 | 9 | 75 | 3 | 19 |
| GSTP1*A/B | 102 | 35 | 55 | 32 | 26 | 31 | 3 | 25 | 8 | 50 |
| GSTP1*A/C | 30 | 10 | 19 | 11 | 10 | 12 | 0 | 0 | 3 | 19 |
| GSTP1*A/D | 0 | 0 | 0 | 0 | 0 | 0 | 0 | 0 | 0 | 0 |
| GSTP1*B/B | 22 | 8 | 18 | 11 | 9 | 11 | 0 | 0 | 1 | 6 |
| GSTP1*B/C | 11 | 4 | 6 | 4 | 1 | 1 | 0 | 0 | 1 | 6 |
| GSTP1*C/C | 1 | 0 | 0 | 0 | 0 | 0 | 0 | 0 | 0 | 0 |
| GSTP1*C/D | 0 | 0 | 0 | 0 | 0 | 0 | 0 | 0 | 0 | 0 |
| TOTAL | 288 | 100 | 170 | 100 | 83 | 100 | 12 | 100 | 16 | 100 |
| GSTP1 Allele | | | | | | | | | | |
| Frequencies | | | | | | | | | | |
| GSTP1*A | 376 | 65 | 215 | 63 | 111 | 67 | 21 | 88 | 17 | 53 |
| GSTP1*B | 157 | 27 | 94 | 28 | 45 | 27 | 3 | 12 | 11 | 34 |
| GSTP1*C | 43 | 7.5 | 31 | 9 | 11 | 7 | 0 | 0 | 4 | 13 |
| GSTP1*D | 0 | 0 | 0 | 0 | 0 | 0 | 0 | 0 | 0 | 0 |
| TOTAL | 576 | 100 | 340 | 100 | 166 | 100 | 24 | 100 | 32 | 100 |
| | Medullo- | | Neuro- | | Osteo- | | Retino- | | Rhabdo- | |
| | blastoma | | blastoma | | sarcoma | | blastoma | | myosarcoma | |
| | (n) | (%) | (n) | (%) | (n) | (%) | (n) | (%) | (n) | (%) |
| GSTM1 Genotypes | | | | | | | | | | |
| GSTM1*0/0 | 8 | 57 | 3 | 30 | 2 | 17 | 4 | 50 | 11 | 64 |
| GSTM1*A/unknown | 3 | 21 | 3 | 30 | 5 | 42 | 2 | 25 | 3 | 18 |
| GSTM1*A/B | 0 | 0 | 1 | 10 | 0 | 0 | 0 | 0 | 0 | 0 |
| GSTM1*B/unknown | 3 | 21 | 3 | 30 | 5 | 42 | 2 | 25 | 3 | 18 |
| TOTAL | 14 | 100 | 10 | 100 | 12 | 101 | 8 | 100 | 17 | 100 |
| GSTM3 Genotypes | | | | | | | | | | |
| GSTM3*A/A | 6 | 67 | 5 | 56 | 7 | 58 | 6 | 86 | 10 | 71 |
| GSTM3*A/B | 3 | 33 | 4 | 44 | 5 | 42 | 1 | 14 | 4 | 29 |
| GSTM3*B/B | 0 | 0 | 0 | 0 | 0 | 0 | 0 | 0 | 0 | 0 |
| TOTAL | 9 | 100 | 9 | 100 | 12 | 100 | 7 | 100 | 14 | 100 |
| GSTT1 Genotypes | | | | | | | | | | |
| GSTT1*0/0 | 3 | 25 | 4 | 44 | 2 | 17 | 0 | 0 | 2 | 14 |
| GSTT1*1/unknown | 9 | 75 | 5 | 56 | 10 | 83 | 7 | 100 | 12 | 86 |
| TOTAL | 12 | 100 | 9 | 100 | 12 | 100 | 7 | 100 | 14 | 100 |
| GSTP1 Genotypes | | | | | | | | | | |
| GSTP1*A/A | 6 | 60 | 4 | 45 | 5 | 42 | 3 | 38 | 5 | 31 |
| GSTP1*A/B | 1 | 10 | 3 | 33 | 4 | 33 | 3 | 38 | 6 | 38 |
| GSTP1*A/C | 2 | 20 | 0 | 0 | 0 | 0 | 1 | 12 | 3 | 19 |
| GSTP1*A/D | 0 | 0 | 0 | 0 | 0 | 0 | 0 | 0 | 0 | 0 |
| GSTP1*B/B | 0 | 0 | 1 | 11 | 3 | 25 | 1 | 12 | 0 | 0 |
| GSTP1*B/C | 1 | 10 | 1 | 11 | 0 | 0 | 0 | 0 | 2 | 13 |
| GSTP1*C/C | 0 | 0 | 0 | 0 | 0 | 0 | 0 | 0 | 0 | 0 |
| GSTP1*C/D | 0 | 0 | 0 | 0 | 0 | 0 | 0 | 0 | 0 | 0 |
| TOTAL | 10 | 100 | 9 | 100 | 12 | 100 | 8 | 100 | 16 | 101 |
| GSTP1 Allele | | | | | | | | | | |
| Frequencies | | | | | | | | | | |
| GSTP1*A | 15 | 75 | 11 | 61 | 14 | 58 | 10 | 63 | 19 | 59 |
| GSTP1*B | 2 | 10 | 6 | 33 | 10 | 42 | 5 | 31 | 8 | 25 |
| GSTP1*C | 3 | 15 | 1 | 6 | 0 | 0 | 1 | 6 | 5 | 16 |
| GSTP1*D | 0 | 0 | 0 | 0 | 0 | 0 | 0 | 0 | 0 | 0 |
| TOTAL | 20 | 100 | 18 | 100 | 24 | 100 | 16 | 100 | 32 | 100 |

TABLE 8
Multivariate Analysis of Pediatric Cancer Risk
for GSTM1 and GSTT1 Genotypes

| | | | Relative | Confidence |
|---|---|---|---|---|
| Disease | Effect | p-Value | risk | interval |
| Acute | GSTM1* A0 vs. 00 | <0.001 | 5.661 | 2.581, 12.415 |
| Lymphoblastic | GSTM1* B0 vs. 00 | 0.001 | 4.278 | 1.795, 10.195 |
| Leukemia | GSTT1* 10 vs. 00 | 0.035 | 2.592 | 1.068, 6.289 |
| Glial Brain | GSTM1* A0 vs. 00 | 0.009 | 4.865 | 1.487, 15.921 |
| Tumors | | | | |
| Osteosarcoma | GSTM1* A0 vs. 00 | 0.038 | 6.900 | 1.116, 42.653 |
| | GSTM1* B0 vs. 00 | 0.002 | 16.000 | 2.775, 92.244 |

1. A high throughput assay for detecting GST alleles present in a patient comprising the steps of:
obtaining a biological sample from the patient;
isolating genomic DNA from the sample;
performing PCR amplification of a portion of the DNA to detect GSTM1 alleles;
performing PCR amplification of a portion of the DNA to detect GSTM3 and GSTT1 alleles;
performing PCR amplification of a portion of the DNA to detect GSTP 1 polymorphisms; and
detecting GSTM1, GSTM3, GSTT1, and GSTP1 polymorphic alleles in the DNA obtained from the PCR amplification steps.

2-7. (canceled)

8. The method of claim 1, wherein the step of performing PCR amplification of a portion of the DNA to detect GSTM1 alleles comprises performing fluorescent, allele-specific PCR using GSTM1-specific primer sequences.

9. The method of claim 8, wherein individual GSTM1-specific primer sequences are selected from SEQ ID NO: 1, SEQ ID NO: 2, SEQ ID NO: 3, SEQ ID NO: 4, and SEQ ID NO: 5.

10. (canceled)

11. The method of claim 8, wherein the portion of the DNA is also PCR-amplified to detect β-actin as a reaction control using B3-actin-specific PCR primer sequences.

12. The method of claim 11, wherein the individual β-actin-specific primer sequences are selected from SEQ ID NO: 6, SEQ ID NO: 7, and SEQ ID NO: 8.

13. (canceled)

14. The method of claim 1, wherein the step of performing PCR amplification of a portion of the DNA to detect GSTM3 and GSTT1 alleles comprises performing PCR using GSTM3- and GSTT1-specific primer sequences.

15. The method of claim 14, wherein the individual GSTM3- and GSTT1-specific primer sequences are selected from SEQ ID NO: 9, SEQ ID NO: 10, SEQ ID NO: 11, SEQ ID NO: 12, SEQ ID NO: 24, SEQ ID NO: 25, and SEQ ID NO: 26.

16. (canceled)

17. The method of claim 1, wherein the step of performing PCR amplification of a portion of the DNA to detect GSTP1 polymorphisms comprises performing fluorescent, allele-specific PCR using GSTP1-specific primer sequences.

18. The method of claim 17, wherein the individual GSTP1-specific primer sequences are selected from SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22 and SEQ ID NO: 23.

19. (canceled)

20. The method of claim 1, wherein the step of detecting GSTM1, GSTM3, GSTT1, and GSTP1 polymorphic alleles in the DNA obtained from the PCR amplification steps includes combining the DNA obtained from the PCR amplification steps to detect GSTM1, GSTM3, GSTT1, and GSTP1 alleles.

21. The method of claim 20, wherein the step of detecting GSTM1, GSTM3, GSTT1 and GSTP1 polymorphic alleles in the DNA obtained from the PCR amplification steps further includes conducting a gel electrophoresis of the combined DNA.

22. (canceled)

23. The method of claim 20, wherein the step of detecting GSTM1, GSTM3, GSTT1 and GSTP1 polymorphic alleles in the DNA obtained from the PCR amplification steps further includes conducting a capillary electrophoresis of the combined DNA.

24. (canceled)

25. The method of claim 20, wherein the step of detecting GSTM1, GSTM3, GSTT1, and GSTP1 polymorphic alleles in the DNA obtained from the PCR amplification steps is followed by performing a long range PCR assay of a portion of the DNA to distinguish GSTM1*A/A or GSTM1*B/B homozygotes from GSTM1*A/null and GSTM1*B/null heterozygotes.

26. The method of claim 25, wherein the step of performing a long range PCR assay of a portion of the DNA is conducted using GSTM1*0-specific primer sequences.

27. The method of claim 26, wherein the GSTM1*0-specific primer sequences are SEQ ID NO: 27 and SEQ ID NO: 28.

28. The method of claim 20, wherein the step of detecting GSTM1, GSTM3, GSTT1 and GSTP1 polymorphic alleles in the DNA obtained from the PCR amplification steps is followed by performing a long range PCR assay of a portion of the DNA to determine the gene dosage of GSTT1.

29. The method of claim 28, wherein the step of performing a long range PCR assay of a portion of the DNA to determine the gene dosage of GSTT1 is conducted using GSTT1*0-specific primer sequences.

30. The method of claim 29, wherein the GSTT1*0-specific primer sequences are SEQ ID NO: 33 and SEQ ID NO: 34.

31. The method of claim 28, wherein the step of performing a long range PCR assay of a portion of the DNA to determine the gene dosage of GSTT1 is conducted using GSTT1*0-specific primer sequences and GSTT1/GSTT2-non-specific primer sequences.

32. The method of claim 31, wherein the GSTT*0-specific primer sequences are SEQ ID NO: 33 and SEQ ID NO: 34 and the GSTT1/GSTT2-non-specific primer sequences are SEQ ID NO: 31 and SEQ ID NO: 32.

33. The method of claim 1, wherein the steps of performing PCR amplification of a portion of the DNA are followed by the steps of identifying portions of the DNA which failed PCR amplification and performing single nucleotide extension verification assays on the portions of the DNA which failed PCR amplification.

34. The method of claim 1, wherein the step of performing PCR amplification of a portion of the DNA to detect GSTM1 alleles includes using primers having the sequences of SEQ ID NO: 1, SEQ ID. NO: 3 and SEQ ID NO: 4; the step of performing PCR amplification of a portion of the DNA to detect GSTM3 and GSTT1 alleles includes using primers having the sequences of SEQ ID NO: 9, SEQ ID NO: 11, SEQ ID NO: 24 and SEQ ID NO: 25; and performing PCR amplification of a portion of the DNA to detect GSTP1 polymorphisms includes using primers having the sequences of SEQ ID NO: 13, SEQ ID NO: 15, SEQ ID NO: 17, SEQ ID NO: 19, SEQ ID NO: 21 and SEQ ID NO: 23.

35. The method of claim 34 wherein the steps of performing PCR amplification of a portion of the DNA are followed by the steps of identifying portions of the DNA which failed PCR amplification and performing single nucleotide extension verification assays on the portions of the DNA which failed PCR amplification.

36. A method of assessing the potential toxicity of chemotherapy in a patient comprising the steps of:
obtaining a biological sample from the patient;
isolating genomic DNA from the sample;
performing PCR amplification of a portion of the DNA to detect GSTM1 alleles;
performing PCR amplification of a portion of the DNA to detect GSTM3 and GSTT1 alleles;
performing PCR amplification of a portion of the DNA to detect GSTP1 polymorphisms;
detecting GSTM 1, GSTM3, GSTT 1, and GSTP1 polymorphic alleles in the DNA obtained from the PCR amplification steps; and
comparing the GSTM1, GSTM3, GSTT1 and GSTP1 polymorphic alleles present to predetermined standards to evaluate the potential toxicity of chemotherapy to the patient.

37-70. (canceled)


SEQUENCE LISTING (47 sequences)

SEQ ID NO: 1 - DNA, 31 bp, Artificial
  M1F GSTM1 Forward primer
gtttcttctgcttcacgtgttatgaaggttc

SEQ ID NO: 2 - DNA, 23 bp, Artificial Sequence
  M1F GSTM1 Forward primer without non-specific sequence tail added to promote completion of non-templated nucleotide addition.
tgcttcacgtgttatgaaggttc

SEQ ID NO: 3 - DNA, 20 bp, Artificial Sequence
  M1R-A GSTM1 Reverse Primer
ttgggaaggcgtccaagcac

SEQ ID NO: 4 - DNA, 23 bp, Artificial Sequence
  M1R-B GSTM1 Reverse primer.
tctttgggaaggcgtccaagcag

SEQ ID NO: 5 - DNA, 20 bp, Artificial Sequence
  M1R-B GSTM1 Reverse primer without non-specific sequence tail added to promote completion of non-templated nucleotide addition.
ttgggaaggcgtccaagcag

SEQ ID NO: 6 - DNA, 19 bp, Artificial Sequence
  ACTB-A Beta-Actin Forward primer.
cctccctggagaagagtac

SEQ ID NO: 7 - DNA, 26 bp, Artificial Sequence
  ACTB-B Beta-Actin Reverse primer.
gtttctgtgttggcgtacaggtcttt

SEQ ID NO: 8 - DNA, 20 bp, Artificial Sequence
  ACTB-B Beta-Actin Reverse primer without non-specific sequence tail added to promote completion of non-templated nucleotide addition.
gtgttggcgtacaggtcttt

SEQ ID NO: 9 - DNA, 26 bp, Artificial Sequence
  M3F GSTM3 Forward primer.
gtttctcctcagtacttggaagagct

SEQ ID NO: 10 - DNA, 20 bp, Artificial Sequence
  M3F GSTM3 Forward primer without non-specific sequence tail added to promote completion of non-templated nucleotide addition.
cctcagtacttggaagagct

SEQ ID NO: 11 - DNA, 26 bp, Artificial Sequence
  M3R GSTM3 Reverse primer.
gtttctcacatgaaagccttcaggtt

SEQ ID NO: 12 - DNA, 20 bp, Artificial Sequence
  M3R GSTM3 Reverse primer without non-specific sequence tail added to promote completion of non-templated nucleotide addition.
cacatgaaagccttcaggtt

SEQ ID NO: 13 - DNA, 25 bp, Artificial Sequence
  P1-104FA GSTP1 Forward primer.
gtttctgacctccgctgcaaataca

SEQ ID NO: 14 - DNA, 19 bp, Artificial Sequence
  P1-104FA GSTP1 Forward primer without non-specific sequence tail added to promote completion of non-templated nucleotide addition.
gacctccgctgcaaataca

SEQ ID NO: 15 - DNA, 28 bp, Artificial Sequence
  P1-104FG GSTP1 Forward primer.
gtttctcttgacctccgctgcaaatacg

SEQ ID NO: 16 - DNA, 19 bp, Artificial Sequence
  P1-104FG GSTP1 Forward primer without non-specific sequence tail added to promote completion of non-templated nucleotide addition.
gacctccgctgcaaatacg

SEQ ID NO: 17 - DNA, 23 bp, Artificial Sequence
  P1-104R GSTP1 Reverse primer.
gtttctcagcccaagccacctga

SEQ ID NO: 18 - DNA, 18 bp, Artificial Sequence
  P1-104R GSTP1 Reverse primer without non-specific sequence tail added to promote completion of non-templated nucleotide addition.
tcagcccaagccacctga

SEQ ID NO: 19 - DNA, 27 bp, Artificial Sequence
  P1-113FT GSTP1 Forward primer.
gtttctctttggtgtctggcaggaggt

SEQ ID NO: 20 - DNA, 17 bp, Artificial Sequence
  P1-113FT GSTP1 Forward primer without non-specific sequence tail added to promote completion of non-templated nucleotide addition.
ggtgtctggcaggaggt

SEQ ID NO: 21 - DNA, 23 bp, Artificial Sequence
  P1-113FC GSTP1 Forward primer.
gtttctggtgtctggcaggaggc

SEQ ID NO: 22 - DNA, 17 bp, Artificial Sequence
  P1-113FC GSTP1 Forward primer without non-specific sequence tail added to promote completion of non-templated nucleotide addition.
ggtgtctggcaggaggc

SEQ ID NO: 23 - DNA, 19 bp, Artificial Sequence
  P1-113R GSTP1 Reverse primer.
tggtctcccacaatgaagg

SEQ ID NO: 24 - DNA, 23 bp, Artificial Sequence
  T1F GSTT1 Forward primer.
ttccttactggtcctcacatctc

SEQ ID NO: 25 - DNA, 27 bp, Artificial Sequence
  T1R GSTT1 Reverse primer.
gtttctacagactggggatggatggtt

SEQ ID NO: 26 - DNA, 22 bp, Artificial Sequence
  T1R GSTT1 Reverse primer without non-specific sequence tail added to promote completion of non-templated nucleotide addition.
tacagactggggatggatggtt

SEQ ID NO: 27 - DNA, 26 bp, Artificial Sequence
  M2F10 forward primer.
aagacagaggaagggtgcatttgata

SEQ ID NO: 28 - DNA, 24 bp, Artificial Sequence
  M5R16A primer sequence.
acagacattcattcccaaagcgac

SEQ ID NO: 29 - DNA, 22 bp, Artificial Sequence
  TPA7SF primer sequence.
ggaagtacagctcagagttctg

SEQ ID NO: 30 - DNA, 22 bp, Artificial Sequence
  TPAREV primer sequence.
agcgggacgaatccgatttcag

SEQ ID NO: 31 - DNA, 22 bp, Artificial Sequence
  GSTT1*A-F forward primer sequence.
aatgctttgtggactgctgagg

SEQ ID NO: 32 - DNA, 21 bp, Artificial Sequence
  GSTT1*A-R Reverse primer sequence.
tgatgcatgtgagtgctgtgg

SEQ ID NO: 33 - DNA, 22 bp, Artificial Sequence
  GSTT1*0-F Forward primer sequence.
tacagttgtgagccaccgtacc

SEQ ID NO: 34 - DNA, 21 bp, Artificial Sequence
  GSTT1*0-R Reverse primer sequence.
atagttgctggccccctcatt

SEQ ID NO: 35 - DNA, 20 bp, Artificial Sequence
  M1nest2-F Forward primer sequence.
tgcttcacgtgttatggagg

SEQ ID NO: 36 - DNA, 24 bp, Artificial Sequence
  M1nest2-R Reverse primer sequence.
catgcgagttattctgtgtgtagc

SEQ ID NO: 37 - DNA, 42 bp, Artificial Sequence
  SNEM1-F Forward primer sequence.
gtttctgtttctgtttctgtttcaccgtatatttgagcccaa

SEQ ID NO: 38 - DNA, 20 bp, Artificial Sequence
  SNEM1-F Forward primer sequence without non-specific tail.
caccgtatatttgagcccaa

SEQ ID NO: 39 - DNA, 35 bp, Artificial Sequence
  SNEM1-R Reverse Primer sequence.
gtttctgtttctgtttctgggaaggcgtccaagca

SEQ ID NO: 40 - DNA, 18 bp, Artificial Sequence
  SNEM1-R Reverse primer sequence without non-specific tail.
tgggaaggcgtccaagca

SEQ ID NO: 41 - DNA, 30 bp, Artificial Sequence
  GSTP1 104F Forward primer sequence.
gtttctgtttcggacctccgctgcaaatac

SEQ ID NO: 42 - DNA, 19 bp, Artificial Sequence
  GSTP1 104F Forward primer sequence without non-specific tail.
ggacctccgctgcaaatac

SEQ ID NO: 43 - DNA, 26 bp, Artificial Sequence
  GSTP1 104R Reverse primer sequence.
gtttctgttgttgtagatgagggaga

SEQ ID NO: 44 - DNA, 20 bp, Artificial Sequence
  GSTP1 104R Reverse primer without non-specific tail.
gttgttgtagatgagggaga

SEQ ID NO: 45 - DNA, 18 bp, Artificial Sequence
  GSTP1 113F Forward primer sequence.
gtggtgtctggcaggagg

SEQ ID NO: 46 - DNA, 22 bp, Artificial Sequence
  GSTP1 113 R Reverse primer sequence.
tctcacatagtcatccttgccc

SEQ ID NO: 47 - DNA, 20 bp, Artificial Sequence
  GSTP1 113R Reverse primer sequence without non-specific tail.
tcacatagtcatccttgccc